(19) United States
(12) Patent Application Publication
(10) Pub. No.: US 2002/0197189 A1
(43) Pub. Date: Dec. 26, 2002

(54) Solar-power battery air freshening device with motor

(76) Inventor: Edgardo R. Lua, Quezon City (PH)

Correspondence Address: Edgardo R. Lua, 245 Banawe St., Cor. Sgt. Rivera, Quezon City, PH

(21) Appl. No.: 10/185,524

(22) Filed: Jun. 26, 2002

Related U.S. Application Data
(60) Provisional application No. 60/302,089, filed on Jun. 26, 2001.

(51) Int. Cl. A62B 7/08; A61L 9/00

(52) U.S. Cl. 422/124; 422/305; 422/306

(57) ABSTRACT

A solar-power battery air freshening device with motor for providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened. The inventive device includes a solar-power battery attached to a housing which contains a motor, a circulating fan, a container which has fragrance emitting substance, air vents on the housing. The solar-power battery is a device which absorbs the energy emitted from sunlight or light from regular light fixtures and transforms the energy into power. The housing is a formed or injected container made of elastic or molded material. The motor is a machine that transforms energy into mechanical energy. The fragrance is the scent emitted by pleasant odor producing substance. The fan is a molded or injected component structure which is designed to move or circulate air when in operation. The container is a molded, injected, or stamped structure normally used to hold substances in solid or liquid form.

### BACKGROUND OF THE INVENTION

1. Field of the Invention

The present invention relates generally to air freshening device and more specifically it relates to a solar-power battery air freshening device with motor for providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened.

2. Description of the Prior Art

It can be appreciated that air freshening device have been in use for years. Typically, air freshening device are comprised of U.S. Pat. No. 5,342,584, an air freshener device and cartridge with battery as means of power. U.S. Pat. No. 3,711,023, an air freshener device utilizing an air conditioning system which releases the evaporated volatile substance into the air to produce an odor. U.S. Pat. No. 3,990,848, an air flow induction device air freshening apparatus with a fan mounted in the housing. A fan driven by a motor is connected with the battery. U.S. Pat. No. 4,647,428, a room air freshening device that uses light bulb for vaporization of liquid. It works when the light bulb is turned on to heat and vaporize fragrance to permeate the room. U.S. Pat. No. 4,808,347, an air freshening apparatus which is plugged into a standard cigarette lighter socket of vehicles, utilizing an electric motor within the housing with a propeller fastened to its shaft.

The main problem with conventional air freshening device are that as in U.S. Pat. Nos. 5,342,584 and 3,990,848, the devices need the usage of batteries. The utilization of batteries to power the devices need more attention to the operation, need the constant change of batteries, add more cost to the device because of the cost of batteries. Another problem with conventional air freshening device are that, as in U.S. Pat. No. 3,711,023, the device uses the air conditioning system to release the evaporated volatile substance into the air to produce an odor. The utilizatiion of the air conditioning system makes the device depended on the time the air conditioning system is working. Another problem with conventional air freshening device are that, as in U.S. Pat. No. 4,647,428, the room freshener device utilizes a light bulb that is plugged to the electrical system. The light bulb has to be turned on and at the same time consumes electricity to function. Another problem with existing products is that, as in U.S. Pat. No. 4,808,347, the air freshening device is plugged into a standard cigarette lighter socket in a vehicle. Again, the device is dependent on another device as in this case the cigarette lighter, which on occasion may not be functioning.

While these devices may be suitable for the particular purpose to which they address, they are not as suitable for providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened. The main problem with conventional air freshening device are that, as in U.S. Pat. Nos. 5,342,584 and 3,990,848, the devices need the usage of batteries. The utilization of batteries to power the devices need more attention to the operation, need the constant change of batteries, add more cost to the device because of the cost of batteries. Another problem is that, as in U.S. Pat. No. 3,711,023, the device uses the air conditioning system to release the evaporated volatile substance into the air to produce an odor. The utilizatiion of the air conditioning system makes the device depended on the time the air conditioning system is working. Also, another problem is that, as in U.S. Pat. No. 4,647,428, the room freshener device utilizes a light bulb that is plugged to the electrical system. The light bulb has to be turned on and at the same time consumes electricity to function.Another problem with existing products is that, as in U.S. Pat. No. 4,808,347, the air freshening device is plugged into a standard cigarette lighter socket in a vehicle. Again, the device is dependent on another device as in this case the cigarette lighter, which on occasion may not be functioning.

In these respects, the solar-power battery air freshening device with motor according to the present invention substantially departs from the conventional concepts and designs of the prior art, and in so doing provides an apparatus primarily developed for the purpose of providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened.

### SUMMARY OF THE INVENTION

In view of the foregoing disadvantages inherent in the known types of air freshening device now present in the prior art, the present invention provides a new solar-power battery air freshening device with motor construction wherein the same can be utilized for providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened.

The general purpose of the present invention, which will be described subsequently in greater detail, is to provide a new solar-power battery air freshening device with motor that has many of the advantages of the air freshening device mentioned heretofore and many novel features that result in a new solar-power battery air freshening device with motor which is not anticipated, rendered obvious, suggested, or even implied by any of the prior art air freshening device, either alone or in any combination thereof.

To attain this, the present invention generally comprises a solarpower battery attached to a housing which contains a motor, a circulating fan, a container which has fragrance emitting substance, air vents on the housing. The solar-power battery is a device which absorbs the energy emitted from sunlight or light from regular light fixtures and transforms the energy into power. The housing is a formed or injected container made of elastic or molded material. The motor is a machine that transforms energy into mechanical energy. The fragrance is the scent emitted by pleasant odor producing substance. The fan is a molded or injected component structure which is designed to move or circulate air when in operation. The container is a molded, injected, or stamped structure normally used to hold substances in solid or liquid form.

There has thus been outlined, rather broadly, the more important features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a solarpower battery air freshening device with motor that will overcome the shortcomings of the prior art devices.

An object of the present invention is to provide a solar-power battery air freshening device with motor for providing a device with solar-power battery utilizing the energy derived from natural sunlight or light emitted from regular room light fixture. The solar-power battery is operationaly connected to a motor wherein a fan is connected to the motor's shaft. The circular movement of the fan moves fragrance emitted from the volatile substance of the fragrance out of the housing to the room or vehicle being freshened.

Another object is to provide a solar-power battery air freshening device with motor that will solve the disadvantages of prior arts wherein they either need to be manually operated, need to utilize batteries, need to be plugged to electrical socket.

Another object is to provide a solar-power battery air freshening device with motor that utilizes natural sunlight or light emitted by regular room light fixtures.

Another object is to provide a solar-power battery air freshening device with motor that it is self contained. It does not need anymore manual operations, does not need anymore batteries, does not need to be plugged into electrical current. All the device needs is ample natural sunlight or light emitted by regular room light fixtures.

Another object is to provide a solar-power battery air freshening device with motor that it is economical. There are no added cost incurred for the solar power cell operated air freshener to operate other than the fragrance which in all air freshener is also an additional component of the whole air freshener device.

Another object is to provide a solar-power battery air freshening device with motor that the invention need minimal maintenace. There are no batteries to change, nothing to manually operate,no need to create electrical system for the invention to plug in. The invention works safely while unattended.

Another object is to provide a solar-power battery air freshening device with motor that employs a circulating fan connected to the motor's shaft. Said motor is driven by the energy derived from the solar-power cell. The fans circulating motion moves fragranced air out of the device to freshen any room or vehicle.

Another object is to provide a solar-power battery air freshening device with motor that is usable in any rooms or vehicles where ample sunlight or light emitted from regular room light fixture.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a perspective view of the invention.

FIG. 2 is a side view of the invention.

FIG. 3 is a perpective view of the cover.

### DESCRIPTION OF THE PREFERRED EMBODIMENT

Turning now descriptively to the drawings, in which similar reference characters denote similar elements throughout the several views, the attached figures illustrate a solar-power battery air freshening device with motor, which comprises a solar-power battery attached to a housing which contains a motor, a circulating fan, a container which has fragrance emitting substance, air vents on the housing. The solar-power battery is a device which absorbs the energy emitted from sunlight or light from regular light fixtures and transforms the energy into power. The housing is a formed or injected container made of elastic or molded material. The motor is a machine that transforms energy into mechanical energy. The fragrance is the scent emitted by pleasant odor producing substance. The fan is a molded or injected component structure which is designed to move or circulate air when in operation. The container is a molded, injected, or stamped structure normally used to hold substances in solid or liquid form.

The solar-power battery is a device which absorbs the energy emitted from sunlight or light from regular light fixtures and transforms the energy into power. As shown in FIGS. through 1 to 2, the solar-power cell is also called solar battery. Its structure utilizes no chemical reaction to produce electricl power and it has no moving parts. Its main function is to absorb sunlight or light from light fixutres to be converted into electrical energy. Solar cells can be arranged into large groupings which may composed of many thousand of individual cells. Because it has no moving parts, it does not require service or fuel. The basic structure and principle of a solar cell can be describe as light enters the device through a layer of material called the antireflection layer which absorbs and traps the light falling on the solar cell and transmits this light into the energy layers of the solar cell which usually are made of silicon oxides or titanium dioxide. The conversion of light into electrical enery called photovoltaic effect occurs in the three energy-conversion layers namely the top junction layer, absorber layer, and the back junction layer. The solar cell also has two additional layers called the electrical contact layers. The layers allow electric current to flow in and out of the cell where light enters. There also the back electrical layer which functions as an electrical contact and conductor. When light falls on the cell, free electrons occur as a result of the interaction of the light with the absorber layer where the energy of an electron increases from a ground state energy to an excited energy state. This results in and energetic,free electrons. The electrons are collected by the electrical contact layers for use in external circuit where they can do useful work. Also know as semiconductors. The semiconductors can absorb light to the thickness of one-hundredth of a centimeter or less. When the electron is lifted to its excited state by consuming light energy, it moves to the external circuit to the electric field and it is in this external circuit that the electron will dissipate its excess energy in some devices as a motor. When the energy is expended, when the solar cell produces electric current to the motor, the above mentioned process starts all over again. The solar-power cell may be arranged into groupings which may be composed of thousands of cells and can function as power stations. Solar cells convert sunlight into electrical energy that can be used in industrial,commercial,and residential applications. As in comsumer products such as electronic toys,hand held calculators as in this invention, to power a motor as an air freshener.

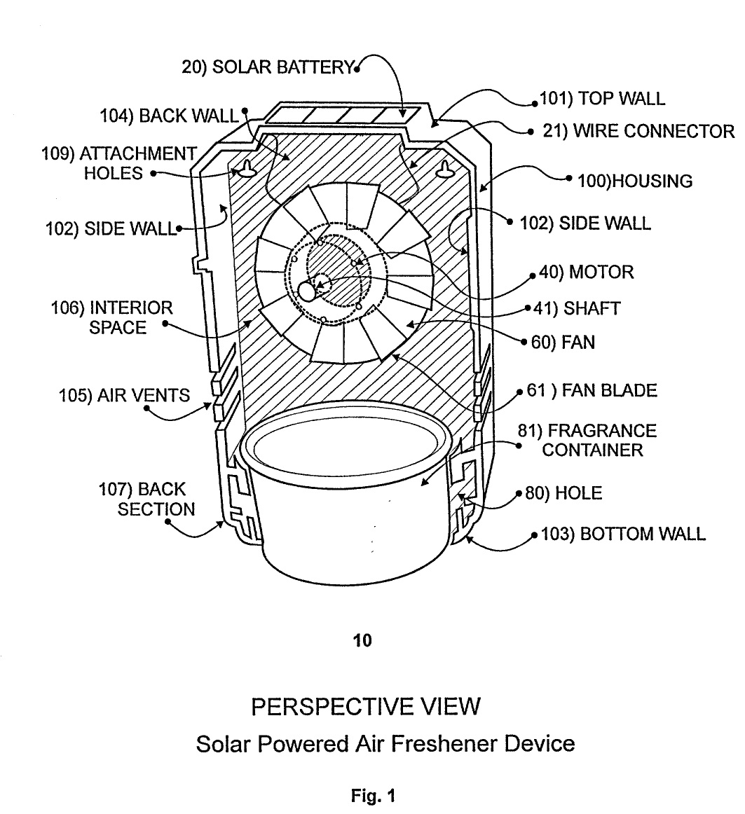

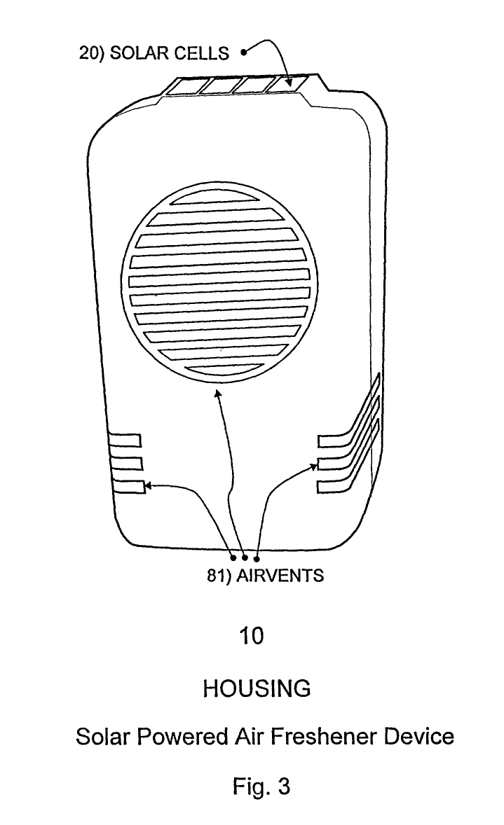

The housing is a formed or injected container made of elastic or molded material. The housing as shown in FIGS. 1 to 3, is a molded, injected, or stamped container made of materials that can be shaped into various forms to hold other objects, parts or component of a product, device or apparatus. The housing may also be constructed from various substances and shaped into numerous forms to contain or hold many kinds of objects, devices, or apparatus into one whole component part which can be adhered to or connected to other components or object.

The motor is a machine that transforms energy into mechanical energy. The circuit is a path which provides continous passage of electricity. It usually contains a driving coil, transistor capacitor. When an electric current is fed to said driving coil from the power source, a magnetic field is generated which reacts repulsively or attractively to magnetic pole arrangements of a magnet. A circuit is composed of a network of transistors, transformers, capacitors, connecting wires and other electronic components within a single device.

The fragrance is the scent emitted by pleasant odor producing substance. The coil is circular or cylindrical shaped object made of current-carrying wire designed to produce a magnetic field. or desinged to provide a resistance or inductance in a circuit. Coils are usually relative to functions of electrical in nature. It is composed of winded current-carrying wire designed to produce magnetic field normally by placing a soft iron core within to produce an electromagnetic field when activated. Coils are usually relative to functions of electrical in nature. It is composed of winded current-carrying wire designed to to produce magnetic field normally by placing a soft iron core within to produce an electromagnetic field when activated.

The fan is a molded or injected component structure which is designed to move or circulate air when in operation. Fan is a device for stirring air. It can come in different sizes and shapes and can drive air movement with a to and fro, oscillating movement depending on the design of the device. It can be made of various materials to include paper, fabric,plastic, metal to name a few and can be manufactured through injection, moulding, and stamping. There are quite a few designs to a fan. It can be straight, bent, formed into a propeller design and many others shapes and dimensions. The main feature of which is that the device can stir or move air usually connected to another device that aids its oscillating motion or circular or rotary motion.

The container is a molded, injected, or stamped structure normally used to hold substances in solid or liquid form. Container is any device or apparatus that will hold another device, object or substance. The conatiner comes in a multitude of shapes and configuration and design and can be constructed with various injectable, mouldable and stampable materials such as paper, plastic, in plates, metal or glass. It's main objective is to hold other materials or substances.

The interconnection of my invention starts with the solar-power battery being attached to the housing of the device. The solor-power battery has a negative and positive polarity wherein electrical wires are connected. The wires are in turn connected inside the housing to the motor which is firmly set in place in the interior of the housing utilizing the housing design to snugly fit the motor. The motor has a shaft connected to the fan. The container has the fragrance medium and is strategically positioned close to the fan so that the volatile vapor of the fragrance will easily move towards the vents of the housing. The solar-power battery can be attached to the housing. The solar-power battery can be positioned in such a manner that it will be able to absorb the light or energy source. This position can be on the top or on the sides of the housing directly or indirectly facing the light or energy source. The connection of the circuit, which connects the solar-power cell with electric wire can be positioned within the housing in any direction where optimum placement of the fan which is connected to the bracket is closest to the fragrance container. In this manner, the agitation or the volatile vapor of the fragrance eminating from the container can be achieved. The fragrance air is forced out of the housing freshening the room or vehicle.

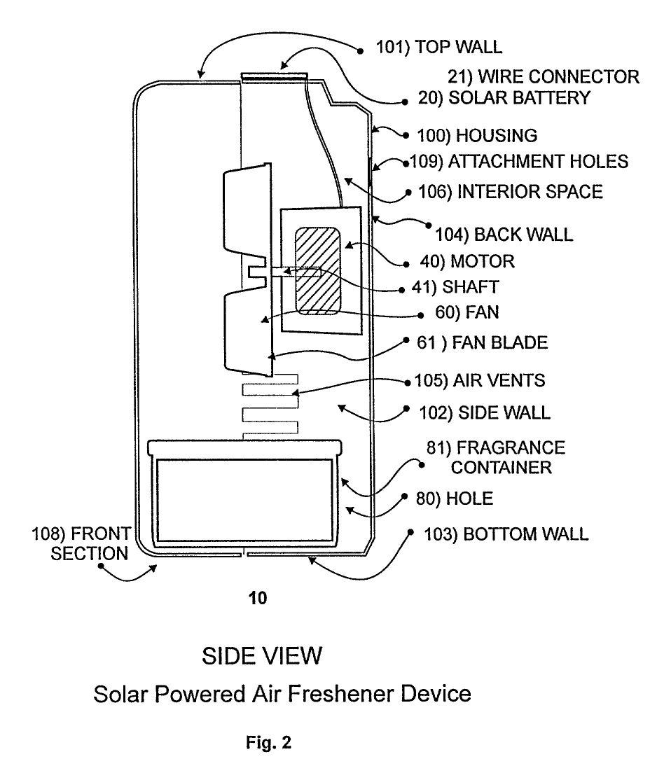

Referring now to FIGS. 1 through 2 and particularly to FIG. 1, a solar-power driven air freshener according to the present invention is shown. The air freshener has a rectangular box shaped housing 100 preferable made of durable, electrically non-conductive material such as polypropylene. Preferably the housing 100 is fabricated by injection molding. As may be seen best by referring to FIG. 1, the interior space of the rectangular box shaped housing 100 contains an electric motor 40. The electric motor 40 has a cylindrical shape which fits coaxially within the hollow interior space 106 of the housing 100, and an output motor shaft 41 extending forward from the front face of the motor 40. Motor shaft 41 is coaxially aligned within the hollow interior space 106 of the housing 100. This is accomplished by choosing the outer diameter of the motor 40 so as to make the outer circumferential surface of the motor 40 fit snugly within the housing 100. As shown in FIG. 1, the solar-power battery 20 wire connectors 21 are connected to the electrical contact end of the motor 40 which is snugly fit inside the housing 100. As shown in FIGS. 1 and 2, the hollow interior space 106 of the rectangular box shaped housing 100 accomodates a fan 60 attached to the motor shaft 41 of the electrical motor 40. Air vents 105 are strategically provided on the housing 100 to permit free movement of air moved by the circular rotation of the fan 60.The back section 107 of the housing 100 has a cover that fits snugly on the front section 108 of the hollow housing 100 wherein air vent 105 can also be located to allow air passage to and from the housing 100. The air freshener includes a container 81 whose dimension fit snugly into the lower hollow section of the housing 100. The container 81 which is made of a material that could hold fragrance made of a substance that would blend into a fragrance medium set in place within the container 81. The back section 107 of the housing 100 and the front section 108 cover of the housing 100 fit perfectly which provide a convenient means for taking off the front cover 108 of the air freshener without tools and for replacing container 100 after its scent has dissipated. Also described previously, the novel design of the solar-power battery air freshener according to the present invention allows easy removal and replacement of container 100 without requiring tools. As illustrated in the back wall 104 of the of the back housing 100, attachment holes 109 are made to optionally combine with the air frehener which provides a convienient means of securing the housing 100 of the air freshener on the wall of the rooms inside a building with screws. Likewise, double sided adhesive tape is also adhered to the back outer side of the housing 100 to provide another adhesive means of the solar-power air freshener on walls or surfaces of rooms or vehicles.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. The construction of a solar-power air freshener device comprising an open front casing having a top wall, opposed side walls, a back wall, and a bottom wall, said bottom wall having a hole provided thereon for insertion of a fragrance container containing scented materials, a fan motor with fan blades attached thereon being secured on the back wall above the fragrance container and a solar battery mounted on said top wall and electronically connected to said fan motor.
